SEQUENCE LISTING (20 sequences)

SEQ ID NO: 1 - protein, 473 aa, Homo sapiens
MRAGPGPTVTLALVLAVAWAMELKPTAPPIFTGRPFVVAWDVPTQDCGPRLKVPLDLNAFDVQASPNEGFVNQNITIFYRDRLGLYPRFDSAGRSVHGGVPQNVSLWAHRKMLQKRVEHYIRTQESAGLAVIDWEDWRPVWVRNWQDKDVYRRLSRQLVASRHPDWPPDRIVKQAQYEFEFAAQQFMLETLRYVKAVRPRHLWGFYLFPDCYNHDYVQNWESYTGRCPDVEVARNDQLAWLWAESTALFPSVYLDETLASSRHGRNFVSFRVQEALRVARTHHANHALPVYVFTRPTYSRRLTGLSEMDLISTIGESAALGAAGVILWGDAGYTTSTETCQYLKDYLTRLLVPYVVNVSWATQYCSRAQCHGHGRCVRRNPSASTFLHLSTNSFRLVPGHAPGEPQLRPVGELSWADIDHLQTHFRCQCYLGWSGEQCQWDHRQAAGGASEAWAGSHLTSLLALAALAFTWTL

SEQ ID NO: 2 - protein, 473 aa, Mus musculus
MRAGLGPIITLALVLEVAWAGELKPTAPPIFTGRPFVVAWNVPTQECAPRHKVPLDLRAFDVKATPNEGFFNQNITTFYYDRLGLYPRFDAAGTSVHGGVPQNGSLCAHLPMLKESVERYIQTQEPGGLAVIDWEEWRPVWVRNWQEKDVYRQSSRQLVASRHPDWPSDRVMKQAQYEFEFAARQFMLNTLRYVKAVRPQHLWGFYLFPDCYNHDYVQNWESYTGRCPDVEVARNDQLAWLWAESTALFPSVYLDETLASSVHSRNFVSFGGREALRVAHTHHANHALPVYVFTRPTYTRGLTGLSQVDLISTIGESAALGSAGVIFWGDSEDASSMETCQYLKNYLTQLLVPYIVNVSWATQYCSWTQCHGHGRCVRRNPSANTFLHLNASSFRLVPGHTPSEPQLRPEGQLSEADLNYLQKHFRCQCYLGWGGEQCQRNYKGAAGNASRAWAGSHLTSLLGLVAVALTWTL

SEQ ID NO: 3 - protein, 509 aa, Homo sapiens
MGVLKFKHIFFRSFVKSSGVSQIVFTFLLIPCCLTLNFRAPPVIPNVPFLWAWNAPSEFCLGKFDEPLDMSLFSFIGSPRINATGQGVTIFYVDRLGYYPYIDSITGVTVNGGIPQKISLQDHLDKAKKDITFYMPVDNLGMAVIDWEEWRPTWARNWKPKDVYKNRSIELVQQQNVQLSLTEATEKAKQEFEKAGKDFLVETIKLGKLLRPNHLWGYYLFPDCYNHHYKKPGYNGSCFNVEIKRNDDLSWLWNESTALYPSIYLNTQQSPVAATLYVRNRVREAIRVSKIPDAKSPLPVFAYTRIVFTDQVLKFLSQDELVYTFGETVALGASGIVIWGTLSIMRSMKSCLLLDNYMETILNPYIINVTLAAKMCSQVLCQEQGVCIRKNWNSSDYLHLNPDNFAIQLEKGGKFTVRGKPTLEDLEQFSEKFYCSCYSTLSCKEKADVKDTDAVDVCIADGVCIDAFLKPPMETEEPQIFYNASPSTLSATMFIVSILFLIISSVASL

SEQ ID NO: 4 - protein, 512 aa, Mus musculus
MGELRFKHLFWGSFVESGGTFQTVLIFLLIPCSLTVDYRAAPILSNTTFLWIWNVPTERCVGNVNDPIDLSFFSLIGSPRKTATGQPVTLFYVDRLGLYPHIDANQAEHYGGIPQRGDYQAHLRKAKTDIEHYIPDDKLGLAIIDWEEWRPTWLRNWKPKDNYRNKSIELVQSTNPGLSITRATQKAIQQLEEAGRKFMEGTLHLGKFLRPNQLWGYYLFPDCYNNKFQDPKYDGQCPAVEKKRNDNLKWLWKASTGLYPSVYLKKDLKSNRQATLYVRYRVVEAIRVSKVGNASDPVPIFVYIRLVFTDRTSEYLLEDDLVNTIGEIVALGTSGIIIWDAMSLAQRAAGCPILHKYMQTTLNPYIVNVTLAAKMCSQTLCNEKGMCSRRKESSDVYLHLNPSHFDIMLTETGKYEVLGNPRVGDLEYFSEHFKCSCFSRMTCKETSDVKNVQDVNVCVGDNVCIKAKVEPNPAFYLLPGKSLLFMTTLGHVLYHLPQDIFVFPRKTLVSTP

SEQ ID NO: 5 - protein, 435 aa, Homo sapiens
MAAHLLPICALFLTLLDMAQGFRGPLLPNRPFTTVWNANTQWCLERHGVDVDVSVFDVVANPGQTFRGPDMTIFYSSQLGTYPYYTPTGEPVFGGLPQNASLIAHLARTFQDILAAIPAPDFSGLAVIDWEAWRPRWAFNWDTKDIYRQRSRALVQAQHPDWPAPQVEAVAQDQFQGAARAWMAGTLQLGRALRPRGLWGFYGFPDCYNYDFLSPNYTGQCPSGIRAQNDQLGWLWGQSRALYPSIYMPAVLEGTGKSQMYVQHRVAEAFRVAVAAGDPNLPVLPYVQIFYDTTNHFLPLDELEHSLGESAAQGAAGVVLWVSWENTRTKESCQAIKEYMDTTLGPFILNVTSGALLCSQALCSGHGRCVRRTSHPKALLLLNPASFSIQLTPGGGPLSLRGALSLEDQAQMAVEFKCRCYPGWQAPWCERKSMW

SEQ ID NO: 6 - protein, 462 aa, Mus musculus
MLGLTQHAQKVWRMKPFSPEVSPGSSPATAGHLLRISTLFLTLLELAQVCRGSVVSNRPFITVWNGDTHWCLTEYGVDVDVSVFDVVANKEQSFQGSNMTIFYREELGTYPYYTPTGEPVFGGLPQNASLVTHLAHTFQDIKAAMPEPDFSGLAVIDWEAWRPRWAFNWDSKDIYRQRSMELVQAEHPDWPETLVEAAAKNQFQEAAEAWMAGTLQLGQVLRPRGLWGYYGFPDCYNNDFLSLNYTRQCPVFVRDQNDQLGWLWNQSYALYPSIYLPAALMGTEKSQMYVRHRVQEALRVAIVSRDPHVPVMPYVQIFYEMTDYLLPLEELEHSLGESAAQGVAGAVLWLSSDKTSTKESCQAIKAYMDSTLGPFIVNVTSAALLCSEALCSGHGRCVRHPSYPEALLTLNPASFSIELTHDGRPPSLKGTLSLKDRAQMAMKFRCRCYRGWRGKWCDKRGM

SEQ ID NO: 7 - protein, 481 aa, Homo sapiens
MKVLSEGQLKLCVVQPVHLTSWLLIFFILKSISCLKPARLPIYQRKPFIAAWNAPTDQCLIKYNLRLNLKMFPVIGSPLAKARGQNVTIFYVNRLGYYPWYTSQGVPINGGLPQNISLQVHLEKADQDINYYIPAEDFSGLAVIDWEYWRPQWARNWNSKDVYRQKSRKLISDMGKNVSATDIEYLAKVTFEESAKAFMKETIKLGIKSRPKGLWGYYLYPDCHNYNVYAPNYSGSCPEDEVLRNNELSWLWNSSAALYPSICVWKSLGDSENILRFSKFRVHESMRISTMTSHDYALPVFVYTRLGYRDEPLFFLSKQDLVSTIGESAALGAAGIVIWGDMNLTASKANCTKVKQFVSSDLGSYIANVTRAAEVCSLHLCRNNGRCIRKMWNAPSYLHLNPASYHIEASEDGEFTVKGKASDTDLAVMADTFSCHCYQGYEGADCREIKTADGCSGVSPSPGSLMTLCLLLLASYRSIQL

SEQ ID NO: 8 - protein, 481 aa, Mus musculus
MQLLPEGQLRLCVFQPVHLTSGLLILFILKSISSLKPARLPVYQRKPFIAAWNAPTDLCLIKYNLTLNLKVFQMVGSPRLKDRGQNVVIFYANRLGYYPWYTSEGVPINGGLPQNTSLQVHLKKAAQDINYYIPSENFSGLAVIDWEYWRPQWARNWNTKDIYRQKSRTLISDMKENISAADIEYSAKATFEKSAKAFMEETIKLGSKSRPKGLWGYYLYPDCHNYNVYATNYTGSCPEEEVLRNNDLSWLWNSSTALYPAVSIRKSFADSENTLHFSRFRVRESLRISTMTSQDYALPVFVYTQLGYKEEPLLFLSKQDLISTIGESAALGAAGIVVWGDMNLTSSEENCTKVNRFVNSDFGSYIINVTRAAEVCSRHLCKNNGRCVRKTWKAAHYLHLNPASYHIEASEDGEFIVRGRASDTDLAVMAENFLCHCYEGYEGADCREMTEASGPSGLSLSSSSVITLCLLVLAGYQSIQL

SEQ ID NO: 9 - protein, 483 aa, Homo sapiens
  VARIANT 230, 231, 232, 233, 234, 235, 236, 237, 238, 239: Xaa = Any Amino Acid
MCNPWVAWLGVLPLFILLTQAALKPAMPPVIKSQPFNIFWAAPTMYCMPSFNVDINLQVFNIISNPLETQSGSKIAIFYPNELGYYPYLSQDGKSFNGGIPQNVSLSEHLRKTADDIGEGVPWWRSEELVVIDWESWKPQWDRNQGSRIIYKNHSLAFTRNHHPYWSEMKVETVAREEFENAGKNFMNITLTLALEMRPKCLWGFYLYPDCYNYDYRINPETYTGNCPNXXXXXXXXXXWLWKKSAALYPSIHLDKILKSSLWALKFVHYRVREAMRVAENARHDYVLPVFIFSRPPYLHSTEALSQVQLVHAIGESAALGRAAGVILWGGYEYSASEHCLSVQQSIRGPLGHYAVNVTSAAKLCSQSLCWWHGRYFEKHLSPPSICICLKAVVRNNFKNKSFRFIISENNKQKTITDMKNGFVCRCYYGHHGPSCHDHSSDLLRVMNKAPTINFNLLVFLIMASSVILLKKILALTTNPIFS

SEQ ID NO: 10 - protein, 494 aa, Mus musculus
MFIQMVYQLGELVLFVLLVAPAALKPAMPFVIKDHPFNVFNAAPTLFCKDNFNVNNMLQVFNIIPNPPETQSGSTITVFYFKELGYYPFFSKDGTSFYGGIPQKVWLSEHLRKSAGDIADAVTLWRSFGLAVIDWEGWRPQWDRNWGSRMIYKMHSLAFTRHHHPDWAETKVRTAAQKEFENAGRSFNNVTLTLALEMRPKRLWGFYLYPDCYNYDYRIMPEPYTGSCPDDEIFRNDQLMWLMEKSAALYPSIYLSKILKSWLNALKFVHFRVREALRVAEMARKDYVLPVFIFSRFTYLQSIEALSEEDLVHTIGESAALGAAGIILWGGYEYSDTKETCLSVRQTVHGLLGPYVLNVTSAAKLCSQWLCWSHGRCVRKTPESSFYLHNPEDSHKNYVSKKGFRFVIPSPSKLKTINNWKNGFVCKCYYGWHGDSCRSHSPNLQKNKAFASGLNSAVIVGWALFVILMNYFPIPYYNGNFSLKPLKRRKIIFL

SEQ ID NO: 11 - protein, 417 aa, Homo sapiens [sequence omitted]

SEQ ID NO: 12 - protein, 412 aa, Mus musculus
MIMHLGLMMVVGLTLCLMHGQALLQVPEHPFSVVWNVPSARCKAHFGVHLPLDALGIVANHGQHFHGQNISIFYKNQFGLYPYFGPRGTAHNGGIPQAVSLDHHLARAAHQILHSLGSSFAGLAVLDWEEWYPLWAGNWGPHRQVYLAASWVWTQQMFPGLDPQEQLHKAHTSFEQAARALMEYTLQLGRTLRPSGLWGFYRYPACGNGWHKMASNYTGHCHAAITTQNTQLRWLWAASSALFPSIYLPPRLPLAYRQAFVRHRLEEAFRVALLEHSHPLPVLAYSRLTHRSSGRFLSLDDLMQTIGVSAALGTAGVVLWGDLSFSSSEEKCWRLHDYLVGTLGPYVINVTKADMACSHQRCHGHGRCARKDPGQMEAFLHLQPDDSLGAWNSFRCHCYSGWAGPTCLEPKP

SEQ ID NO: 13 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

SEQ ID NO: 14 - protein, 812 aa, Mus musculus
MDHKEVILLFLLLLKPGQGDSLDGYISTQGASLFSLTKKQLAAGGVSDCLAKCEGETDFVCRSFQYHSKEQQCVIMAENSKTSSIIRMRDVILFEKRVYLSECKTGIGNGYRGTMSRTKSGVACQKWGATFPHVPNYSPSTHPNEGLEENYCRNPDNDEQGPWCYTTDPDKRYDYCNIPECEEECMYCSGEKYEGKISKTMSGLDCQAWDSQSPHAHGYIPAKFPSKNLKMNYCRNPDGEPRPWCFTTDPTKRWEYCDIPRCTTPPPPPSPTYQCLKGRGENYRGTVSVTVSGKTCQRWSEQTPHRHNRTPENFPCKNLEENYCRNPDGETAPWCYTTDSQLRWEYCEIPSCESSASPDQSDSSVPPEEQTPVVQECYQSDGQSYRGTSSTTITGKKCQSWAAMFPHRHSKTPENFPDAGLEMNYCRNPDGDKGPWCYTTDPSVRWEYCNLKRCSETGGSVVELPTVSQEPSGPSDSETDCMYGNGKDYRGKTAVTAAGTPCQGWAAQEPHRHSIFTPQTNPRAGLEKNYCRNPDGDVNGPWCYTTNPRKLYDYCDIPLCASASSFECGKPQVEPKKCPGRVVGGCVANPHSWPWQISLRTRFTGQHFCGGTLIAPEWVLTAAHCLEKSSRPEFYKVILGAHEEYIRGSDVQEISVAKLILEPNNRDIALLKLSRPATITDKVIPACLPSPNYMVADRTICYITGWGETQGTFGAGRLKEAQLPVIENKVCNRVEYLNNRVKSTELCAGQLAGGVDSCQGDSGGPLVCFEKDKYILQGVTSWGLGCARPNKPGVYVRVSRFVDWIEREMRNN

SEQ ID NO: 15 - protein, 790 aa, Sus scrofa
DSLDDYVNTQGAFLFSLSRKQVAARSVEECAAKCEAETNFICRAFQYHSKDQQCVVMAENSKTSPIARMRDVVLFEKRIYLSECKTGNGKNYRGTTSKTKSGVICQKWSVSSPHIPKYSPEKFPLAGLEENYCRNPDNDEKGPWCYTTDPETRFDYCDIPECEDECMHCSGEHYEGKISKTMSGIECQSWGSQSPHAHGYLPSKFPNKNLKMNYCRNPDGEPRPWCFTTDPNKRWEFCDIPRCTTPPPTSGPTYQCLKGRGENYRGTVSVTASGHTCQRWSAQSPHKHNRTPENFPCKNLEENYCRNPDGETAPWCYTTDSEVRWDYCKIPSCGSSTTSTEHLDAPVPPEQTPVAQDCYRGNGESYRGTSSTTITGRKCQSWVSMTPHRHEKTPGNFPNAGLTMNYCRNPDADKSPWCYTTDPRVRWEYCNLKKCSETEQQVTNFPAIAQVPSVEDLSEDCMFGNGKRYRGKRATTVAGVPCQEWAAQEPHRHSIFTPETNPRAGLEKNYCRNPDGDDNGPWCYTTNPQKLFDYCDVPQCVTSSFDCGKPKVEPKKCPARVVGGCVSIPHSWPWQISLRYRYRGHFCGGTLISPEWVLTAKHCLEKSSSPSSYKVILGAHEEYHLGEGVQEIDVSKLFKEPSEADIALLKLSSPAVITDKVIPACLPTPNYVVADRTACYITGWGETKGTYGAGLLKEARLPVIENKVCNRYEYLGGKVSPNELCAGHLAGGIDSCQGDSGGPLVCFEKDKYILQGVTSWGLGCALPNKPGVYVRVSRFVTWIEEIMRRN

SEQ ID NO: 16 - protein, 812 aa, Bos taurus
MLPASPKMEHKAVVFLLLLFLKSGLGDLLDDYVNTQGASLLSLSRKNLAGRSVEDCAAKCEEETDFVCRAFQYHSKEQQCVVMAENSKNTPVFRMRDVILYEKRIYLLECKTGNGQTYRGTTAETKSGVTCQKWSATSPHVPKFSPEKFPLAGLEENYCRNPDNDENGPWCYTTDPDKRYDYCDIPECEDKCMHCSGENYEGKIAKTMSGRDCQAWDSQSPHAHGYIPSKFPNKNLKMNYCRNPDGEPRPWCFTTDPQKRWEFCDIPRCTTPPPSSGPKYQCLKGTGKNYGGTVAVTESGHTCQRWSEQTPHKHNRTPENFPCKNLEENYCRNPNGEKAPWCYTTNSEVRWEYCTIPSCESSPLSTERMDVPVPPEQTPVPQDCYHGNGQSYRGTSSTTITGRKCQSWSSMTPHRHLKTPENYPNAGLTMNYCRNPDADKSPWCYTTDPRVRWEFCNLKKCSETPEQVPAAPQAPGVENPPEADCMIGTGKSYRGKKATTVAGVPCQEWAAQEPHQHSIFTPETNPQSGLERNYCRNPDGDVNGPWCYTMNPRKPFDYCDVPQCESSFDCGKPKVEPKKCSGRIVGGCVSKPHSWPWQVSLRRSSRHFCGGTLISPKWVLTAAHCLDNILALSFYKVILGAHNEKVREQSVQEIPVSRLFREPSQADIALLKLSRPAIITKEVIPACLPPPNYMVAARTECYITGWGETQGTFGEGLLKEAHLPVIENKVCNRNEYLDGRVKPTELCAGHLIGGTDSCQGDSGGPLVCFEKDKYILQGVTSWGLGCARPNKPGVYVRVSPYVPWIEETMRRN

SEQ ID NO: 17 - protein, 797 aa, Oryzias latipes
MDFCWKAALLLGAVICSASSSDVEGYSKTEGAWVLSLHRRQYSVNSVTDCAAKCNAETTFTCKSFAYVEKDQECWTAASNSKTEPVLRRQNSALYEKNDYLLECVNGLGTDYRGTKAKTKSGKVCQRWEARFPHRPNITPQTHPRADLDSNFCRNPDGDSRGPWCYTTDPETRWEHCNVTSCGEDCIHCNGEDYRGKASITENGYTCQRWDSQSPHSHGYNPDAIPEKYLEENYCRNPDGDPRPWCFTTSSSKRWDYCAIPRCTSEPPTIVPELTCASGEGQAYRGTVGVTVTGKACQMWSDQTPHKHSRTPENYPCKGLDNNYCRNPDHEKMPWCYTTDPETRWEFCRVPRCGDSPDPDEAVSPPEENNDCYEGNGANYRGVTSETVSGKKCQMWSSMSPHSHDKSPQNFPEAGLRRNFCRNPDGDRAPWCYTTDPKVRWEYCSLKKCSEPTKSPQPQDTQSPAEKDCKIGNGESYRGPTSITVSGVTCQGWRDQSPHTHSSFTPQTHPDKGLEGNECRNPDGDSNGPWCYTTDRNKKWDYCQIPDCAEDLTCGTPVYKPRRCFGRIVGGCQSRPHSWPWQISLRTSSGIHFCGGTLIDPQWVLTAKHCLERSTRPSAYKVLMGIHKERAIEPSKQERRLEKIVQGPIGVDIALLKLDRPADINDKVLPACLPEKDYTVPSDTGCYVTGWGETQGTGGEGVLKETGFPVIENRVCNGPSYLNGRVKSHEMCAGNRDGGHDSCQGDSGGPLVCFSQNKYVVQGVTSWGLGCANAMKPGVYVRVSKFIDWIETTMKAG

SEQ ID NO: 18 - protein, 474 aa, Bos taurus
MGMFRRHHISFRSFAGSSGTPQAVFTFLLLPCCLALDFRAPPLISNTSFLWAWNAPVERCVNRRFQLPPDLRLFSVKGSPQKSATGQFITLFYADRLGYYPHIDEKTGKTVFGGIPQLGNLKSHLEKAKNDIAYYIPNDSVGLAVIDWENWRPTWARNWKPKDVYRDESVELVLQKNPQLSFPEASKIAKVDFETAGKSFMQETLKLGKLLRPNHLWGYYLFPDCYNHNHNQPTYNGNCPDVEKRRNDDLEWLWKESTALFPSVYLNIRLKSTQNAALYVRNRVQEAIRLSKIASVESPLPVFVYARPVFTDGSSTYLSQGDLVNSVGEIVSLGASGIIMWGSLNLSLSVQSCMNLGTYLNTTLNPYIINVTLAAKMCSQVLCHDGGVCTRKHWNSSDYLHLNPMNFAIQTGEGGKYTVPGTLTLEDLQKFSDTFYCSCYSNLSCKKRVDIKNVHSVDVCMAEDVCIDAFLKPP

SEQ ID NO: 19 - protein, 493 aa, Sus scrofa
MGVQRLQHISFRSFFVPSGAPQVVFTFLLIPCCLALDFRASPIIPNTTFLWVWNAPTESCAKKFYMPPDLSLFSFVTSPRASVTGQFLTLFYANRLGYYPHVDENTGKNVNGGIPQLGSLQRHLDKAEKDILHYMQIDKVGLSVIDWENWRPTWERNWKEKAIYRRQSIELVQQKNIKLTPAAATKLAKREFEKAGKTFMQETLKLGKLLRPNHLWGYYLFPDCYNHNYHKPGYNGSCLDIEKRRNDALDWLWKESTALFPSIYLNTRLKPSQVALFVRNRVQEAIRVSKVANAQSPLPVFVYTRPVFSGASSRYLSQDDLVNTIGETVALGASGIVMWGSLNLSLTMQSCMNLGSYLKTTLNPYLINVTLAAKMCSQVLCQEQGVCTRKHWNSSDYLHLNPANFAIRTGKGNKYIVHGKPTLEDLKEFSKNFYCSCFANFHCKERADIENIHAINVCITEDVCVEAFLNSEPELPDEVQQDNQPPCGGSGRC

SEQ ID NO: 20 - protein, 520 aa, Ovis aries
LDFRAPPLISNTSFLWAWNAPAERCVKIFKLPPDLRLFSVKGSPQKSATGQFITLFYADRLGYYPHIDEKTGNTVYGGIPQLGNLKNHLEKAKKDIAYYIPNDSVGLAVIDWENWRPTWARNWKPKDVYRDESVELVLQKNPQLSFPEASKIAKVDFETAGKSFMQETLKLGKLLRPNHLWGYYLFPDCYNHNYNQPTYNGNCSDLEKRRNDDLDWLWKESTALFPSVYLNIKLKSTPKAAFYVRNRVQEAIRLSKIASVESPLPVFVYHRPVFTDGSSTYLSQGDLVNSVGEIVALGASGIIMWGSLNLSLTMQSCMNLGNYLNTTLNPYIINVTLAAKMCSQVLCHDEGVCTRKQWNSSDYLHLNPMNFAIQTGKGGKYTVPGKVTLEDLQTFSDKFYCSCYANINCKKRVDIKNVHSVNVCMAEDICIEGPVKLQPSDHSSSQNEASTTTVSSISPSTTATTVSPCTPEKQSPECLKVRCLEAIANVTQTGCQGVKWKNTSSQSQSSIQNIKNQTTY

(19) United States
(12) Patent Application Publication
(10) Pub. No.: US 2008/0248021 A1
(43) Pub. Date: Oct. 9, 2008

(54) Use of Hyaluronidase in Combination with Plasmin for the Induction of Posterior Vitreous Detachment

(75) Inventor: Lisa R. Grillone, Carlsbad, CA (US)

Correspondence Address: KNOBBE MARTENS OLSON & BEAR LLP, 2040 MAIN STREET, FOURTEENTH FLOOR, IRVINE, CA, 92614, US

(73) Assignee: ISTA PHARMACEUTICALS, INC., Irvine, CA (US)

(21) Appl. No.: 11/993,099

(22) PCT Filed: Jun. 30, 2006
(86) PCT No.: PCT/US2006/026021

§ 371 Date: Jun. 18, 2008

Related U.S. Application Data
(60) Provisional application No. 60/695,897, filed on Jun. 30, 2005.

(51) Int. Cl. A61K 38/47; A61P 27/02

(52) U.S. Cl. 424/94.62

(57) ABSTRACT

The present invention is directed to compositions and processes related to use of hyaluronidase in combination with plasmin for the induction of posterior vitreous detachment.

### RELATED APPLICATIONS

This application claims the benefit of U.S. Provisional Application No. 60/695,897, filed Jun. 30, 2005, the entire disclosure of which is hereby expressly incorporated by reference.

### FIELD OF THE INVENTION

The present invention is directed to compositions and processes related to use of hyaluronidase in combination with plasmin for the induction of posterior vitreous detachment.

### DESCRIPTION OF THE RELATED ART

The adult human eye is a slightly asymmetrical sphere with an approximate sagittal diameter of 24 to 25 mm, a transverse diameter of 24 mm, and a volume of about 6.5 cc. The human eye can be divided into three different layers: an external layer, an intermediate layer and an internal layer. The external layer of the eye consists of the sclera, which is often referred to as the “white of the eye,” and the cornea, which covers the front of the eye. The intermediate layer is divided into an anterior portion and a posterior portion; the anterior portion consists of the circular pigmented iris, the crystalline lens and ciliary body, while the posterior portion consists of the choroid layer. The internal layer consists of the retina, which is the sensory part of the eye. The retina is essentially a layer of nervous tissue, which runs along the inside rear surface of the choroid layer and can be divided into an optic portion and a non-optic portion. The optic portion, which participates in the visual mechanism, contains the rods and cones that are the effectual organs of vision.

The human eye can also be divided into three chambers. The anterior chamber between the cornea and the iris, and the posterior chamber between the iris and the crystalline lens, are filled with aqueous humor. In contrast, the vitreous chamber between the crystalline lens and the retina is filled with a more viscous liquid, called the vitreous (also known as the vitreous body or vitreous humor). The vitreous humor in a normal eye is a clear gel occupying about 80% of the volume of the eyeball. Light that enters the eye through the cornea, pupil, and lens, is transmitted through the vitreous to the retina.

The vitreous humor of a normal human eye is a gel that is roughly 99% water and 1% macromolecules. These macromolecules include a network of collagen fibrils, hyaluronic acid, soluble glycoproteins, sugars and other low molecular weight metabolites. Type II collagen is the principal fibrillar collagen of the vitreous, but the vitreous also contains collagen types V, IX, and XI. The posterior portion of the vitreous body, the posterior hyaloid surface (also known as the posterior vitreous cortex), is in direct contact with the inner retinal surface most prominently at the vitreous base, optic disc, and along the major retinal vessels. Normal adhesion of the vitreous to the retina is mediated by cellular and molecular interactions between the posterior vitreous cortex and the inner limiting membrane (ILM) of the retina. The ILM is essentially the basement membrane of retinal Mueller cells. The ILM contains collagen types I and IV, glycoproteins such as laminin and fibronectin and other glycoconjugates. These components are thought to bridge and bind collagen fibers between the vitreous and the ILM.

With age, the vitreous humor changes from gel to liquid and as it does so it gradually shrinks and separates from the ILM of the retina. This process is known as “posterior vitreous detachment” (PVD) and is a normal occurrence after age 40. However, degenerative changes in the vitreous may also be induced by pathological conditions such as diabetes, Eale's disease and uveitis. Also, PVD may occur earlier than normal in nearsighted people and in those who have had cataract surgery. Usually, the vitreous makes a clean break from the retina. Occasionally, however, the vitreous adheres tightly to the retina in certain places. These small foci of resisting, abnormally firm attachments of the vitreous can transmit great tractional forces from the vitreous to the retina at the attachment site. This persistent tugging by the vitreous often results in horseshoe-shaped tears in the retina. Unless the retinal tears are repaired, vitreous fluid can seep through this tear into or underneath the retina and cause a retinal detachment, a very serious, sight-threatening condition. In addition, persistent attachment between the vitreous and the ILM can result in bleeding from rupture of blood vessels, which results in the clouding and opacification of the vitreous.

The development of an incomplete PVD has an impact on many vitreoretinal diseases including vitreomacular traction syndrome, vitreous hemorrhage, macular holes, macular edema, diabetic retinopathy, diabetic maculopathy and retinal detachment. Thus, an important goal of vitreous surgery is to separate the vitreous from the retina in a manner that prevents vitreous traction.

In order to remove the vitreous from the eye, a microsurgical procedure called vitrectomy is usually performed. In this procedure the vitreous is removed from the eye with a miniature handheld cutting device while simultaneously replacing the removed vitreous with saline solution to prevent collapse of the eye. Surgical removal of the vitreous using this method is highly skill-dependent, and complete removal of the cortical vitreous remains a difficult task. Furthermore, mechanical vitrectomy carries the risk of complications such as scarring, tearing and other damage to the retina. Obviously, such damage is highly undesirable as it can compromise the patient's vision after surgery.

One of the primary aims of vitrectomy is to produce PVD. However, the operation is expensive and requires skillful expertise. PVD can facilitate vitrectomy and decrease the incidence of intraoperative complications. Furthermore, complete PVD may be an important factor to prevent or heal these vitreoretinal diseases. Therefore, safe and simple methods of producing PVD, such as pharmacologic vitreolysis, would provide a practical means of facilitating or even substituting vitrectomy.

### SUMMARY OF THE INVENTION

The present invention provides methods of treating or preventing a disorder, or a complication of a disorder, of the eye of a subject by contacting a vitreous and/or aqueous humor with an effective amount of a composition comprising a hyaluronidase in combination with a plasmin.

The present invention also provides methods of treating or preventing a disorder, or a complication of a disorder, of the eye of a subject by contacting a vitreous and/or aqueous humor with an effective amount of a first composition comprising a hyaluronidase and an effective amount of a second composition comprising a plasmin.

In another aspect, the present invention provides methods of liquefying the vitreous body of a subject by contacting a vitreous and/or aqueous humor with an effective amount of a composition comprising a hyaluronidase in combination with a plasmin.

The present invention also provides methods of liquefying the vitreous body of a subject by contacting a vitreous and/or aqueous humor with an effective amount of a first composition comprising a hyaluronidase and an effective amount of a second composition comprising a plasmin.

In an additional aspect, the present invention provides methods of inducing posterior vitreous detachment in an eye of a subject by contacting a vitreous and/or aqueous humor with an effective amount of a composition comprising a hyaluronidase in combination with a plasmin.

The present invention also provides methods of inducing posterior vitreous detachment in an eye of a subject by contacting a vitreous and/or aqueous humor with an effective amount of a first composition comprising a hyaluronidase and an effective amount of a second composition comprising a plasmin.

The methods of the invention can be practiced independent of vitrectomy, or as an adjunct to vitrectomy.

In a further aspect, the present invention provides methods of performing a vitrectomy in a subject by contacting a vitreous and/or aqueous humor with an effective amount of a composition comprising a hyaluronidase in combination with a plasmin.

The present invention also provides methods of performing a vitrectomy in a subject by contacting a vitreous and/or aqueous humor with an effective amount of a first composition comprising a hyaluronidase and an effective amount of a second composition comprising a plasmin.

In one embodiment, the hyaluronidase is non-recombinant hyaluronidase. In another embodiment, the hyaluronidase is recombinant hyaluronidase. In another embodiment, the plasmin is non-recombinant plasmin. In another embodiment, the plasmin is recombinant plasmin. In another embodiment, the hyaluronidase is a variant of hyaluronidase. In another embodiment, the plasmin is a variant of plasmin.

The methods of the invention can be used to treat or prevent an eye disorder, or a complication of an eye disorder, of a subject, where the disorder of the eye is selected from the group consisting of retinal detachment, retinal tear, vitreous hemorrhage, diabetic vitreous hemorrhage, proliferative diabetic retinopathy, non-proliferative diabetic retinopathy, age-related macular degeneration, macular holes, vitreomacular traction, macular pucker, macular exudates, cystoid macular edema, fibrin deposition, retinal vein occlusion, retinal artery occlusion, subretinal hemorrhage, amblyopia, endophthalmitis, retinopathy of prematurity, glaucoma, retinitis pigmentosa, and any combination thereof.

The methods of the invention can be used to treat or prevent an eye disorder, or a complication of an eye disorder, of a subject by effecting one or more outcomes including, but not limited to, reducing the viscosity of the vitreous, liquefying the vitreous, inducing posterior vitreous detachment, clearing or reducing hemorrhagic blood from the vitreous and/or aqueous humor, clearing or reducing intraocular foreign substances from the vitreous and/or aqueous humor, clearing or reducing materials toxic to the retina from the vitreous and/or aqueous humor, increasing the diffusion of an agent or a composition administered to the vitreous and/or aqueous humor, reducing retinal neovascularization and any combination thereof.

The methods of the invention can be used to treat or prevent an eye disorder, or a complication of an eye disorder, of a subject further by contacting the vitreous and/or aqueous humor with an effective amount of a composition comprising chondroitinase, collagenase, dispase; RGD containing peptides, anti-integrin antibody; P2Y receptor antagonists, urea, hydroxyurea, thiourea, angiogenic inhibitors, VEGF inhibitors, PLGF inhibitors, and any combination thereof.

The methods of the invention can be used to treat or prevent an eye disorder, or a complication of an eye disorder, of a subject, where the composition is a liquid solution, and where the step of contacting the vitreous and/or aqueous humor with the composition comprises injecting the solution into the vitreous and/or aqueous humor.

The present invention also provides a composition comprising at least one hyaluronidase in combination with at least one plasmin.

The present invention further provides a kit comprising a first composition comprising at least one hyaluronidase and a second composition comprising at least one plasmin.

### BRIEF DESCRIPTION OF THE DRAWINGS

FIG. 1. Diagrammatic representation of the putative endoproteolytic processing of Hyal-1. Unprocessed Hyal-1 is shown in the upper figure. After endoproteolytic processing, two fragments are generated that produce two separate N-termini. The 22-amino acid fragment is presumably linked to the rest of the protein by disulfide bonds.

FIG. 2A-D. Alignment of the conceptual translation of the cDNA of all human and mouse hyaluronidase genes identified by 2001 using PIMA program. Identical amino acids are boxed. Conserved blocks, presumably representing regions critical to enzymatic activity, can be seen throughout. Hyal2_h—SEQ ID NO: 1; Hyal2_m—SEQ ID NO: 2; PH20_h—SEQ ID NO: 3; PH20_m—SEQ ID NO: 4; Hyal1_h—SEQ ID NO: 5; Hyal1_m—SEQ ID NO: 6; Hyal4_h—SEQ ID NO: 7; Hyal4_m—SEQ ID NO: 8; Hyalp1_h—SEQ ID NO: 9; Hyalp1_m—SEQ ID NO: 10; Hyal3_h—SEQ ID NO: II; Hyal3_m—SEQ ID NO: 12.

FIG. 3A-B. Alignment of the human PH-20 hyaluronidase, GenBank Accession No. NM_{—}153189 (SEQ ID NO: 3) with bovine (SEQ ID NO: 18), GenBank Accession No. AAP55713; pig sperm adhesion molecule 1, SPAM1 (SEQ ID NO: 19), GenBank Accession No. NP_{—}999176; and ovine testicular hyaluronidase (SEQ ID NO: 20).

FIG. 4. The domain structure of human plasminogen is represented where: K1−K5=the 5 kringle domains, B-CHAIN=catalytic domain of plasmin, and the arrows indicate the sites of proteolytic cleavage by plasmin, elastase, and plasminogen activators (PA'S).

FIG. 5A-B. Alignment of plasminogens from human, GenBank Accession No. NP_{—}000292 (SEQ ID NO: 13), mouse, GenBank Accession No. NP_{—}032903 (SEQ ID NO: 14), pig, GenBank Accession No. P06867 (SEQ ID NO: 15), cow, GenBank Accession No. P06868 (SEQ ID NO: 16), and fish, GenBank Accession No. BAD97814 (SEQ ID NO: 17).

### DETAILED DESCRIPTION OF THE PREFERRED EMBODIMENT

As used herein, the term “substantially complete posterior vitreous detachment” and “substantially complete posterior vitreous separation” refers to separation of the vitreous from the inner limiting membrane to produce a clean vitreoretinal surface devoid of vitreous collagen and free of cortical vitreous remnants. The complete separation or detachment of the vitreous allows the vitreous to fall freely from the eye cup by gravity without the need for mechanical separation. The complete separation of the vitreous results in no vitreous strands remaining attached and a smooth surface of the inner limiting membrane (ILM) as determined by a scanning electron microscope (SEM). Incomplete or partial separation results in collagen fibers remaining attached to the retinal surface that are often visible by SEM.

Unless defined otherwise, technical and scientific terms used herein have the same meaning as commonly understood by one of ordinary skill in the art to which this invention belongs. See, e.g., Dorland's Illustrated Medical Dictionary (30^{th} Edition), D. M. Anderson, P. D. Novak, J. Keith and M. A. Elliott, Eds. Saunders (an Imprint of Elsevier), Philadelphia, Pa., 2003

Definition “treating,” means the reduction or amelioration of any medical disorder to any extent, and includes, but does not require, a complete cure of the disorder.

“Preventing” means to defend or protect against the development of a disorder, i.e., to function as a prophylactic.

“Disorder” means any disease, dysfunction, syndrome, condition, pain, or any combination thereof. Disorder also includes any complications from any disease, dysfunction, syndrome, condition, pain or any combination thereof.

“Subject” means any mammal, particularly a human.

“Contacting” means any mode of administration that results in interaction between a composition and an object being contacted (e.g., vitreous, aqueous humor, etc.). The interaction of the composition with the object being contacted can occur at substantially the same time as the administration of the composition, over an extended period of time starting from around the time of administration of the composition, or be delayed from the time of administration of the composition.

“Composition” means a combination or mixture of one or more substances.

“Substance” means that which has mass and occupies space.

“Foreign substance” means any substance that is determined by a medical-doctor, clinician, veterinarian or researcher to be harmful or toxic to the eye of a subject and/or to be a substance that is not normally found in a healthy mammalian eye.

“Ophthalmologically acceptable carrier” is a substance with which the combination of the invention (hyaluronidase and plasmin) can be combined, without making the combination of the invention unsuitable (as determined by a medical doctor, clinician, veterinarian or researcher) for its intended use in the eye of a subject. Non-limiting examples of an ophthalmologically acceptable carrier include balanced salt solution (BSS).

“Pharmaceutically acceptable carrier” includes, without limitation, water, buffered saline, polyol (for example, glycerol, propylene glycol, liquid polyethylene glycol), or suitable mixtures thereof. Other examples of pharmaceutically acceptable carriers and methods for making such carriers and formulations thereof are found, for example, in Remington's Pharmaceutical Sciences (20th Edition, A. Gennaro (ed.), Lippincott, Williams & Wilkins, 2000).

“An effective amount” means an amount of a substance or composition that elicits a response in an eye of a human or other animal that is being sought by a researcher, veterinarian, medical doctor or other clinician.

“Inducing” means to bring about or stimulate the occurrence of a desired result.

“Reduce” means to decrease to any extent.

“Toxic effects to the eye” means any adverse effect to the eye of a subject that is determined to be harmful to the subject by a researcher, veterinarian, medical doctor or other clinician.

“Vitreous” means the vitreous humor, also referred to as the vitreous body, which occupies the chamber between the crystalline lens of the eye and the retina.

“Stabilizing a protein” means protecting a protein from degradation and/or inactivation through the use of one or more stabilizing agents.

“Hyaluronidase” means any protein made or derived from the amino acid sequence of hyaluronidase.

“Plasmin” means any protein made or derived from the amino acid sequence of plasminogen that has a cleavage of the peptide bond between Arg561 and Val562 of human plasminogen (or counterpart). The cleavage of the peptide bond between Arg561 and Val562 can be accomplished using plasminogen activators. Plasminogen activators include, but are not limited to, streptokinase, staphylokinase, tissue-type plasminogen activator and urokinase.

### Hyaluronidase

Hyaluronidase is a general term used to describe enzymes that are able to breakdown the substrate hyaluronate (hyaluronic acid, hyaluronan), however, some of these enzymes are also able to cleave chondroitin sulphate, albeit at a slower rate (Kreil, G. 1995 Protein Sci. 4:1666-1669).

Hyaluronate (HA) is a linear unsulfated glycosaminoglycan polymer, with an average molecular mass greater than 10,000. The polymer is made up of alternating N-acetylglycosamine and glucuronic acid residues linked by glycosidic bonds, but unlike other glycosaminoglycans, it lacks a covalently linked peptide. High levels of HA are present normally in the joint capsule, in the vitreous of the eye, in Wharton's jelly of the umbilical cord, in amniotic fluid and fetal tissues, and in all tissues undergoing rapid proliferation or repair; 50% of the hyaluronate in the body is found in the skin. Enhanced levels of HA occur in inflammation, edema, the swelling following organ transplantation, stroke, or myocardial infarction, in sepsis, wound repair, and in carcinogenesis. The hyaluronate isolated from various sources has an identical chemical structure (Hynes, W. L. et al. 2000 FEMS Microbiol. Lett. 183:201-207).

The hyaluronidases can be subdivided into three types: I—hyaluronate-4-glycanohydrolases (EC 3.2.1.35, hyaluronoglucosaminase), II—hyaluronate-3-glycanohydrolases (EC 3.2.1.36, hyaluronoglucuronidase), and III—hyaluronate lyases (EC 4.2.2.1) that catalyse the breakdown of HA (Pessini, A. C. et al. 2001 Toxicon 39:1495-1504). Hyaluronate-4-glycanohydrolases are the testicular-type hyaluronidases found in mammalian spermatozoa, lysosomes and the venoms of various insects and snakes. The second group, hyaluronate-3-glycanohydrolases, are produced by leeches, some hookworms and krill (Karlstam, B. et al. 1991 Polar Biol. 11:501-507). Both of these groups of hyaluronidases degrade hyaluronate with the formation of tetrasaccharides as the end product. The third group, hyaluronate lyases, are produced by various bacteria and act as endo-N-acetylhexosaminidases by elimination across the β-1-4 linkage. Unlike the other hyaluronidases, the products of hyaluronate lyases are unsaturated disaccharides (Hynes, W. L. et al. 2000 FEMS Microbiol Lett. 183:201-207). Further, hyaluronate-4-glycanohydrolases and hyaluronate lyases degrade hyaluronate by liberating the endoglucosaminyl group, i.e. they are endohexosaminidases, while leech hyaluronidase liberates endoglucuronyl group, i.e it works like an endo-beta-glucuronidase (Karlstam, B. et al. 1991 Polar Biol. 11:501-507).

I. Hyaluronate-4-glycanohydrolases

In vertebrates, these enzymes are grouped into two classes, the neutral-active hyaluronidases, such as sperm-associated PH-20 and those with an acid pH optimum such as those found in human liver and plasma (Frost, G. I. et al. 1997 Biochem. Biophys. Res. Commun. 236:10-15). The human genome contains six paralogous hyaluronidase-like sequences with approximately 40% identity to each other. They are grouped into two tightly-linked triplets on human chromosomes 3p21.3 (HYAL1, HYAL2 and HYAL3) and 7q31.3 (HYAL4, PH20/SPAM1 and HYALP1). The three hyaluronidase sequences in chromosome 3 have similar genomic structures. HYAL1 has an additional retained intron within exon 1 not present in the other hyaluronidase sequences. The three hyaluronidase genes clustered on chromosome 7 have much larger introns than those on chromosome 3, and contain an additional exon.

The orthologous mouse genes are found on syntenic regions, on mouse chromosomes 9 F1-F2 and 6 A2. The degree of homology between the pairs of orthologs of human and mouse is much greater than that between the six human paralogs (Csoka, A. B. et al. 2001 Matrix Biol. 20:499-508).

### Hyal-1, Plasma Hyaluronidase

The acid-active hyaluronidase in serum was the first one to be purified from mammalian somatic tissues, cloned, sequenced and expressed (Frost, G. I. et al. 1997 Biochem. Biophys. Res. Commun. 236:10-15). The 57-kDa protein is a single polypeptide chain of 49 kDa with approximately 8 kDa of post-translational glycosylation. It is approximately 40% identical and 60% homologous to the enzyme found in sperm, PH-20. The highest levels of mRNA of HYAL1 are found in the major parenchymal organs such as liver, kidney, spleen and heart. The mouse ortholog was also cloned and expressed (Csoka, T. B. et al. 1997 FEBS Lett. 417:307-310) and observed to be 73% identical to the human sequence.

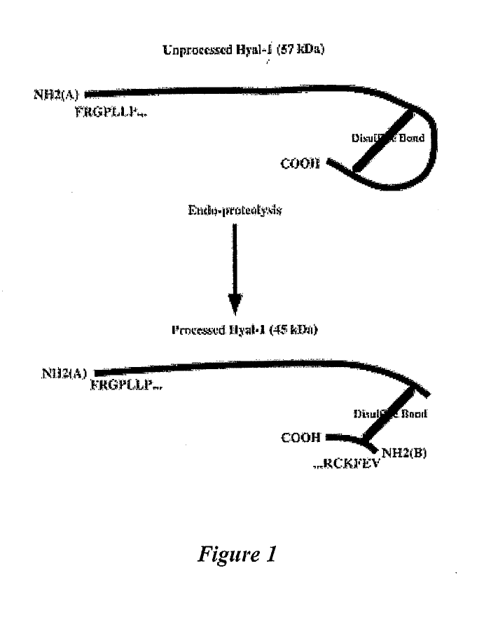

Urine, long known to contain high levels of hyaluronidase activity, was observed to contain Hyal-1 at 100 times the specific activity of that found in plasma. An additional second activity with a molecular weight of 45 kDa is found in urine, and by amino acid sequencing was determined to be Hyal-1 with approximately 100 amino acids deleted from the carboxy region (FIG. 1), resulting in two polypeptide chains bound by disulphide linkages (Csoka, T. B. et al. 1998 Genomics 48:63-70). This does not represent a zymogen/processed enzyme relationship, since the two forms of the enzyme have similar specific activities. The two isozymes of Hyal-1 are also found in cultured cells, with the higher molecular weight isozyme predominant in the culture medium, and the shorter processed isozyme predominant in the cell layer. There are also two forms of PH-20 (Cherr, G. N. et al. 1996 Dev. Biol. 175:142-153; Meyer, M. F. et al. 1997 FEBS Lett. 413:385-388), the higher molecular weight form being a glycosylphosphatidyl-inositol-(GPI-)linked protein attached to the plasma membrane.

### Hyal-2

Another widely expressed and important human acid-active hyaluronidase, Hyal-2, is encoded by a gene at an adjacent chromosomal site to Hyal-1. Hyal-2 has an unusual substrate specificity, cleaving high-molecular-weight HA polymers to intermediate size fragments of approximately 20 kDa. Lung fibroblasts possess an acid-active hyaluronidase in plasma membrane extracts with a similar size specificity for HA as Hyal-2.

### Hyal-3

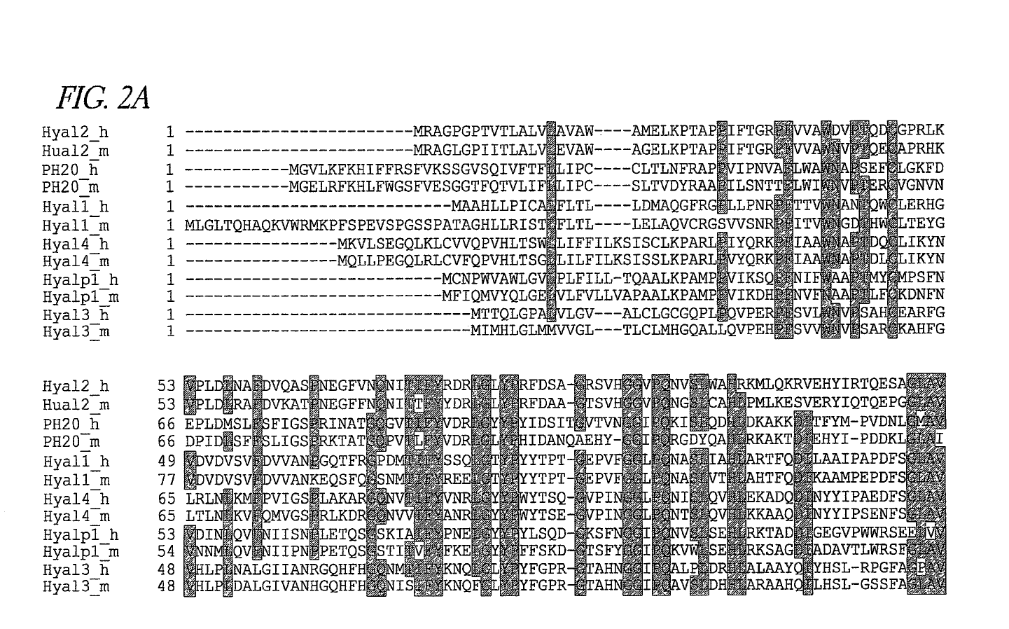

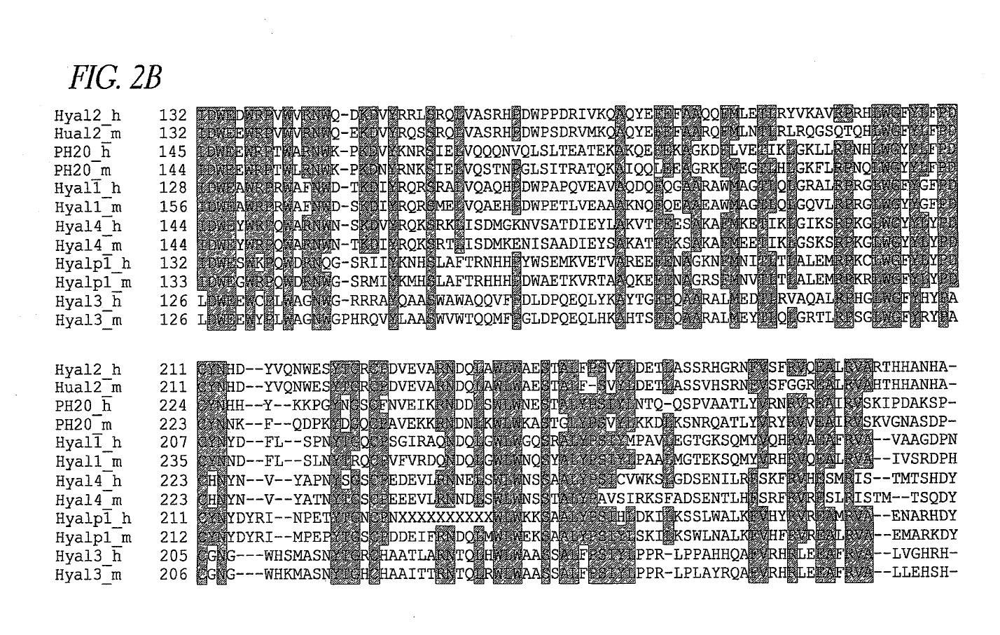

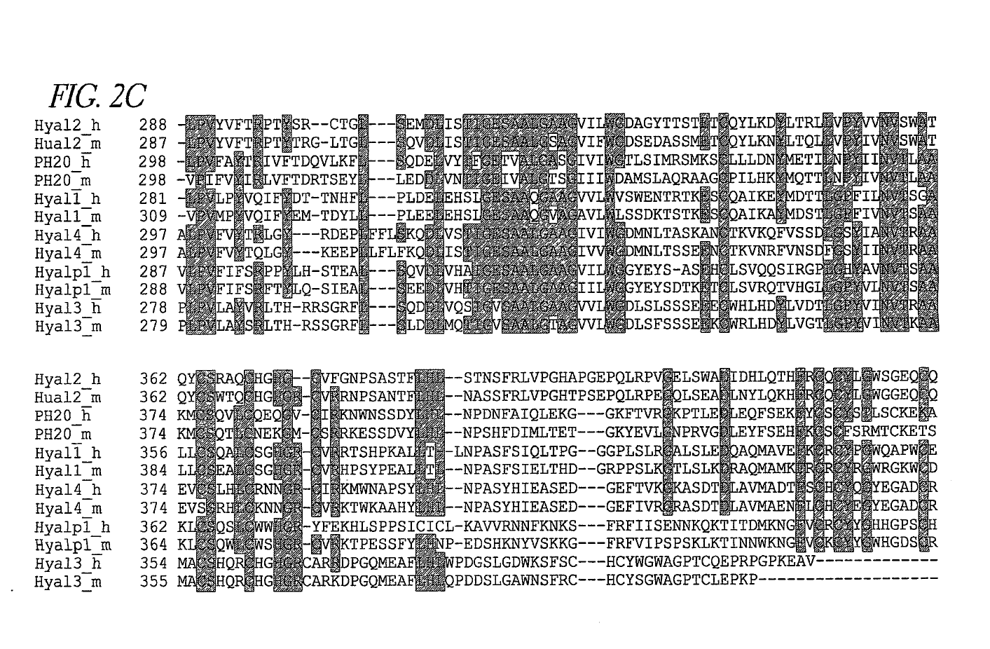

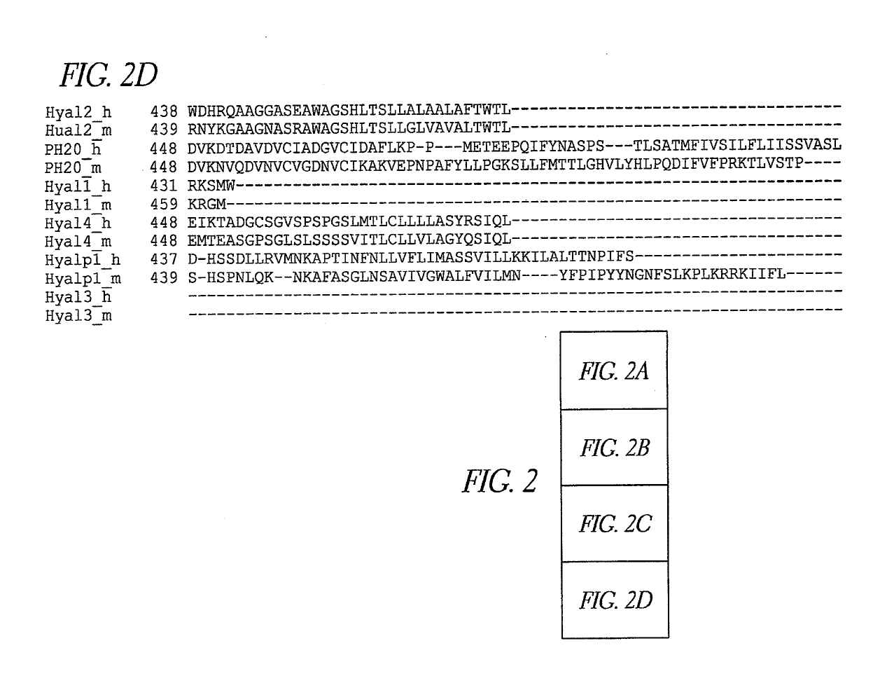

Little is known about Hyal-3, the third enzyme coded for at the 3p21.3 locus. Strong hybridization expression patterns are found in mammalian testis and bone marrow. These two tissues retain a fetal and stem cell-like state for the life of the animal, suggesting that Hyal-3 may be important in stem cell regulation. Mouse Hyal-3 was cloned, and it has approximately 80% amino acid identity to the human sequence (Csoka, A. B. et al. 2001 Matrix Biol. 20:499-508). This degree of identity is higher than that between the human and the mouse Hyal-1 orthologs (73%) (FIG. 2), which suggests that Hyal-3 may have an important function in vivo but so far it has not been conclusively shown to possess hyaluronidase activity in vitro.

### PH-20/SPAM1, Testicular Hyaluronidase

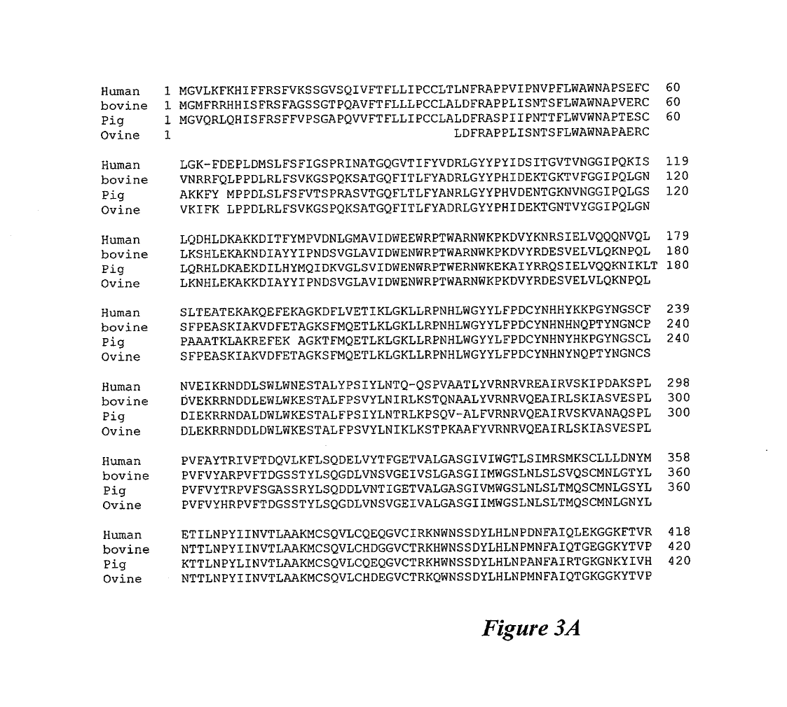

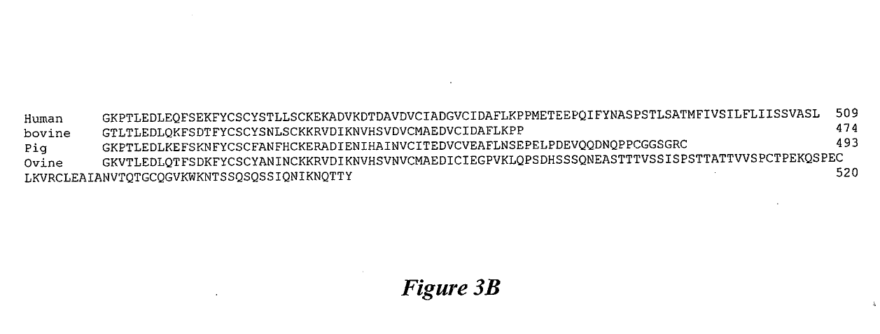

Referring to FIG. 3A-B, the testicular hyaluronidase, PH-20/SPAM1, is important during egg fertilization by sperm (Cherr, G. N. et al. 1996 Dev. Biol. 175:142-153). Expression of SPAM1 has been unanimously reported in the testis in various species: human PH-20 hyaluronidase, GenBank Accession No. NM_{—}153189 (SEQ ID NO: 3); bovine PH-20, GenBank Accession No. AAP55713, (SEQ ID NO: 18); pig sperm adhesion molecule 1, SPAM1, GenBank Accession No. NP_{—}999176, (SEQ ID NO: 19); and ovine testicular hyaluronidase (SEQ ID NO: 20). Expression has also been detected in the human epididymis, vas deferens, prostate and placenta and the murine epididymis, kidney, uterus, vagina and oviduct. Expression of SPAM1 has not been detected in the human female reproductive tract. A catalytic domain has been shown to degrade hyaluronic acid. This molecule is a major extracellular matrix component of the cumulus cell layer that surrounds the ovum, and SPAM1 has been shown to remove this cumulus layer in vitro. SPAM1 has hyaluronic acid and zona pellucida binding regions that are distinct from its catalytic domain and is also involved in an intracellular signalling pathway in sperm cells upon binding to the zona pellucida (Dunn, C. A. et al. 2005 BMC Genomics 6:47).

### Hyal-4

A novel hyaluronidase paralog, termed Hyal-4 is identified at chromosome 7q31.3. The human HYAL4 cDNA is calculated to be 2414 nucleotides in length. Expression of HYAL4 is restricted in placenta and skeletal muscle. Preliminary evidence indicates that Hyal-4 is a chonrdoitinase with no activity against HA. Human and mouse Hyal-4 have 77% amino acid identity (Csoka, A. B. et al. 2001 Matrix Biol. 20:499-508).

### HYALP1

HYALP1 is a pseudogene in humans because two deletions exist that cause premature termination codons. In mouse and possibly other mammals, HYALP1 may encode an active hyaluronidase enzyme in mammals.

Non-Mammalian Hyaluronate-4-glycanohydrolases

Snake, bee and scorpion venoms contain 33-110 kDa hyaluronidases which are active at an in vitro pH range between 4.0 and 6.0 (Pessini, A. C et al. 2001 Toxicon 39:1495-1504).

II. Hyaluronate-3-glycanohydrolases

Hyaluronate-3-glycanohydrolases liberate the endoglucuronyl group of HA, i.e. it works like an endo-beta-glucuronidase. The hyaluronidase purified from Antarctic krill has an optimum pH of 5.3, a temperature optimum of 37° C. and a molecular weight of 80,000 Daltons.

### III. Hyaluronate Lyases

A wide variety of microorganisms produce enzymes capable of degrading hyaluronate. Gram-positive organisms capable of producing hyaluronidase include various species of Streptococcus, Staphylococcus, Peptostreptococcus, Propionibacterium, Streptomyces and Clostridium. Bacteriophages from two species of streptococci, Streptococcus pyogenes and Streptococcus equi, encode hyaluronidase. Eight complete bacterial hyaluronidase genes, and two bacteriophage genes, have had their nucleotide sequence determined. The bacterial hyaluronidases, along with their accession number, are from S. aureus (U21221), Streptococcus agalactiae (Y15903), S. pheumoniae (L20670), S. griseus (AB028210), S. coelicolor (AL031124), P. acnes (U15927), C. perfringens (P26831), Proteus vulgaris (1095454), along with a partial sequence of the lyase from Bacteroides thetaiiotaomicron (L42367). Two sequenced bacteriophage hyaluronidases are both derived from temperate phages that infect group A streptococci (M19348 and U28144) (Hynes, W. L. et al. 2000 FEMS Lett. 183:201-207). Among those hyaluronidases of which the deduced amino acid sequence is known, there is a wide variation in the molecular masses. The streptococcal bacteriophage enzymes are the smallest, ranging between 36 and 40 kDa, depending on the presence or absence of the collagenous motif (Hynes, W. L. et al. 1995 Infect. Immun. 63:3015-3020). The deduced proteins of the other hyaluronidases are much larger: Streptococcus agalactiae 121 kDa, S. pheumoniae 107 kDa, C. perfringens 114 kDa, S. aureus 92 kDa, P. acnes 82 kDa, and the two Streptomyces sp. 77 and 84 kDa (Hynes, W. L. et al. 2000 FEMS Lett. 183:201-207). Molecular masses of other (non-sequenced) hyaluronidases also vary considerably, ranging from 50 to 160 kDa (Linhardt, R. J. et al. 1986 Appl. Microbiol. Biotechnol. 12:135-176). Most of the identified bacterial hyaluronidases are more active at acidic pH (Benchetrit, L. C. et al. 1978 J. Bacteriol. 134:221-228; Brunish, R. et al. 1958 J. Biol. Chem. 231:291-301; Hill, J. 1976 Infect. Immun. 14:726-735; Linder, L. et al. 1971 Scand. J. Dent. Res. 79:528-532; Nord, C. E. 1971 Odontol. Rev. 22:125-136; Rautela, G. S et al. 1973 Arch. Biochem. Biophys. 158:687-694), while peptostreptococcal hyaluronidase with an estimated molecular weight of 160,000 has its optimum pH for activity around neutrality (Tam, Y.-C. et al. 1985 Infect. Immun. 47:508-513).

Hyaluronidases have been employed therapeutically for many years. Hyaluronidases increase tissue membranes permeability, reduce viscosity and render tissues more readily permeable to injecting fluids (spreading effect). Hyaluronidases are widely used in many medical fields, namely orthopedia, surgery, ophthalmology, internal medicine, oncology, dermatology, gynecology, etc. (Menzel and Farr 1998 Cancer Lett. 131:3-11).

### Plasmin

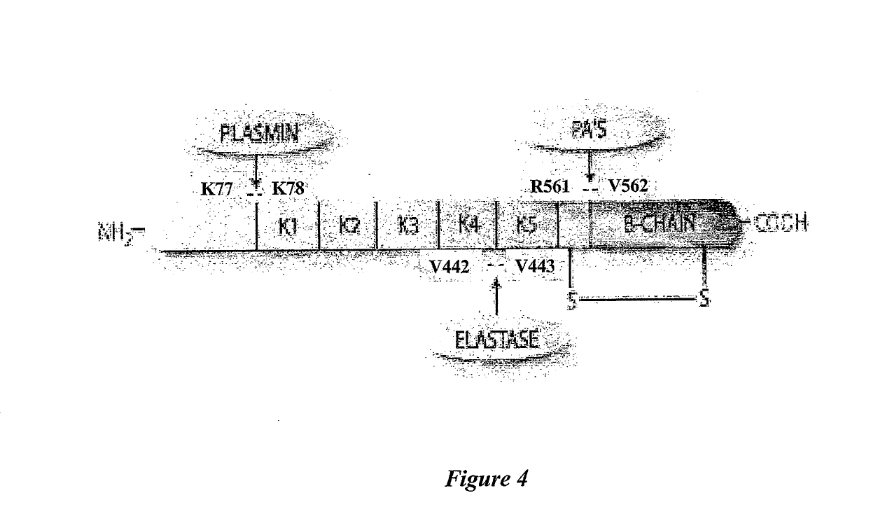

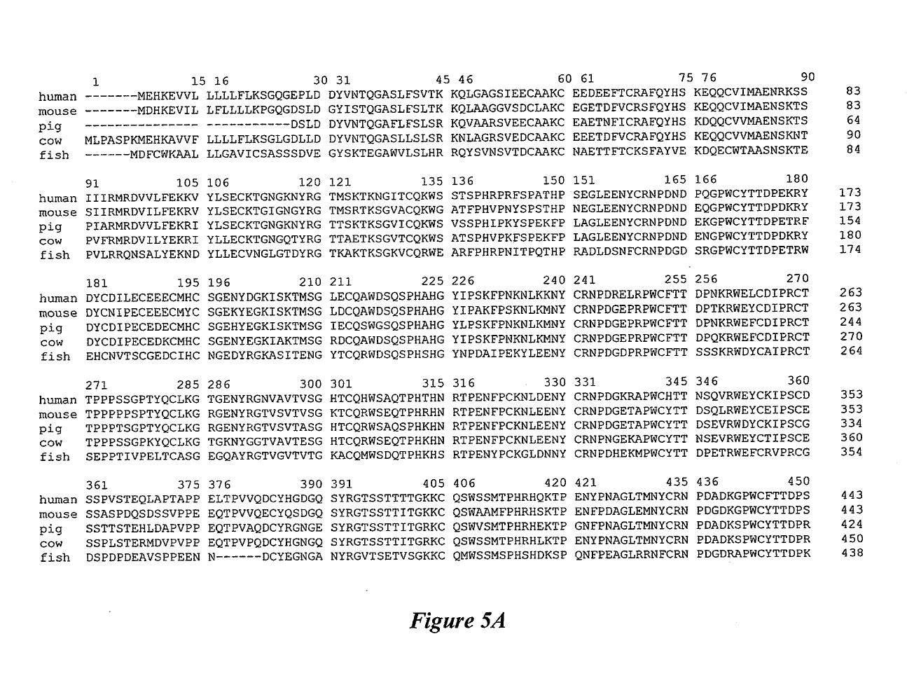

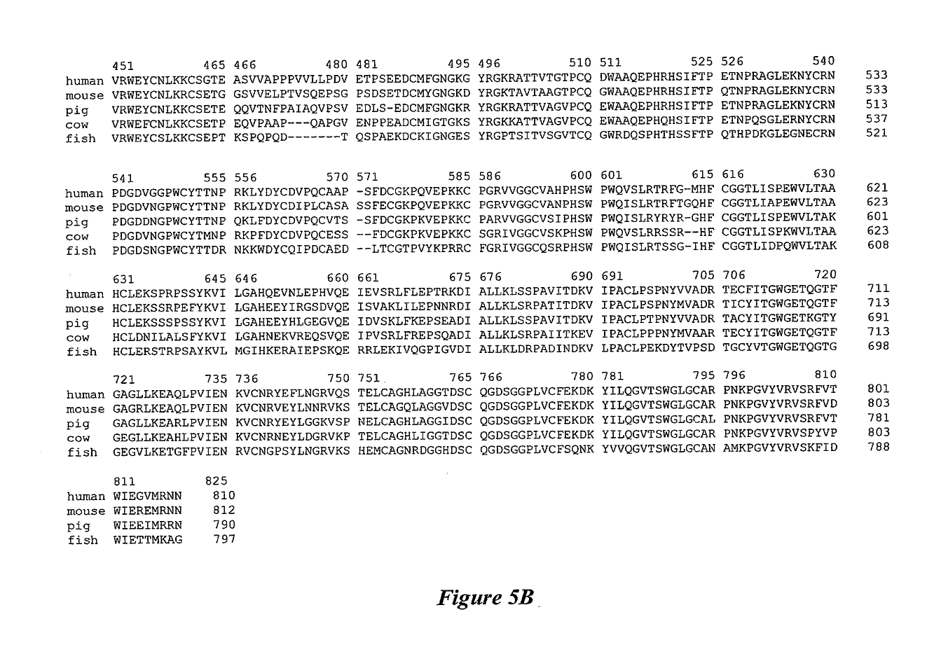

The blood fibrinolytic (plasminogen/plasmin) system comprises an inactive proenzyme, plasminogen, that can be converted to the active enzyme, plasmin, which in turn degrades fibrin into soluble fibrin degradation products. Human plasminogen (SEQ ID NO: 13) is a single-chain glycoprotein with Mᵣ 92,000, present in plasma at a concentration of 1.5 to 2 μM. It consists of 791 amino acids (which together with the signal peptide of 19 amino acids equals a total of 810 amino acids) and contains five homologous triple-loop structures or “kringles” (FIG. 4). These kringles contain structures, called lysine binding sites and aminohexyl binding sites, that mediate the specific binding of plasminogen to fibrin and the interaction of plasmin with α₂-antiplasmin, and thereby play a crucial role in the regulation of fibrinolysis. Other plasminogens known in the art are: mouse (SEQ ID NO: 14), cow (SEQ ID NO: 15), pig (SEQ ID NO: 16), and fish (SEQ ID NO: 17) (FIG. 5A-B).

Native plasminogen (Glu-plasminogen) is easily converted by limited plasmic digestion to modified forms commonly designated “Lys-plasminogen”. This conversion occurs by hydrolysis of the Arg⁶⁸-Met⁶⁹, Lys⁷⁷-Lys⁷⁸, or Lys⁷⁸-Val⁷⁹ peptide bonds. Plasminogen is converted to plasmin by cleavage of the Arg⁵⁶¹-Val⁵⁶² peptide bond. This specific cleavage is mediated by plasminogen activators, e.g. tissue plasminogen activator and urokinase. The plasmin molecule is a two-chain trypsin-like serine proteinase with an active site composed of His⁶⁰³, Asp⁶⁴⁶, and Ser⁷⁴¹ (Lijnen, H. R. 2001 Ann. N.Y. Acad. Sci. 236:226-236). The serine protease function of the plasmin molecule is located in the carboxy-terminal part of the original protein (giving the light chain after the activation cleavage).

Various forms of human plasmin have been described, prepared and isolated, namely Glu¹-plasmin, Lys⁷⁷-plasmin, Val⁴⁴²-plasmin, and Val⁵⁶²-plasmin. Glu¹-plasmin is prepared from the native Glu¹-zymogen in the presence of plasmin inhibitors, Lys⁷⁷-plasmin is prepared from either the native zymogen or the plasmin-degraded Lys⁷⁷-zymogen, and Val⁴⁴²-plasmin is prepared from the elastase-degraded Val⁴⁴²-zymogen. Val⁵⁶²-plasmin, the light (B) chain of Glu¹-plasmin, Lys⁷⁷-plasmin, and Val⁴⁴²-plasmin, is prepared by partial reduction of the two interchain-disulfide bonds connecting the heavy (A) chain and light (B) chain of the larger plasmins. Recombinant plasmin can be prepared from the isolated sulfhydryl forms of the heavy (A) chain and light (B) chain (Robbins, K. C. 1981 Methods Enzymol. 80:379-387).

### Vitreolysis

Pharmacological vitreolysis is a method of using one or more proteinaceous and/or chemical and/or nucleic acid agents to treat or prevent a disorder, or a complication of a disorder, of an eye of a subject. The present invention provides methods of pharmacological vitreolysis using at least one hyaluronidase in combination with at least one plasmin. Specifically, the present invention provides methods of treatment or prevention of eye disorders, or complications of eye disorders, by contacting the vitreous and/or aqueous humor with an effective amount of a composition comprising a hyaluronidase in combination with a plasmin, or with an effective amount of a first composition comprising at least one hyaluronidase and an effective amount of a second composition comprising at least one plasmin. These methods result in outcomes such as, but not limited to, liquefaction of the vitreous, posterior vitreous detachment, reduction or clearing of hemorrhagic blood from the vitreous and/or aqueous humor, reduction or clearing of intraocular foreign substances from the vitreous and/or aqueous humor, increasing diffusion of an agent or composition administered to the vitreous and/or aqueous humor, decreasing extraretinal neovascularization, and any combination thereof. These methods may be used either as an adjunct to vitrectomy, or in the absence of vitrectomy.

Accordingly, the present invention provides, as a first aspect, a method of treating or preventing a disorder, or a complication of a disorder, of the eye of a subject comprising contacting the vitreous and/or aqueous humor with an effective amount of a composition comprising a hyaluronidase in combination with a plasmin.

In a second aspect, the present invention provides a method of treating or preventing a disorder, or a complication of a disorder, of the eye of a subject comprising contacting the vitreous and/or aqueous humor with an effective amount of a first composition comprising a hyaluronidase and an effective amount of a second composition comprising a plasmin. In a further embodiment of this aspect of the invention, the first and second compositions may be administered to a subject at substantially the same time or at different times.

In a third aspect, the present invention provides a method of liquefying the vitreous comprising contacting the vitreous and/or aqueous humor with an effective amount of a composition comprising a hyaluronidase in combination with a plasmin. In one embodiment of this aspect of the invention, the liquefaction of the vitreous decreases the viscosity of the vitreous humor. In other embodiments of the invention, the liquefaction of the vitreous increases the rate of clearance from the vitreous cavity and/or aqueous humor of blood, deposited material, foreign substances and/or materials toxic to the eye, especially the retina. In another embodiment of this aspect of the invention, the liquefaction of the vitreous decreases extraretinal neovascularization. In yet another embodiment of this aspect of the invention, the liquefaction of the vitreous increases the diffusion of an agent or composition administered to the vitreous and/or aqueous humor. In a further embodiment of this aspect of the invention, the liquefaction of the vitreous helps in the removal of the vitreous during standard vitrectomy or 25 Gauge (or smaller) vitrectomy.

In a fourth aspect, the present invention provides a method of liquefying the vitreous comprising contacting the vitreous and/or aqueous humor with an effective amount of a first composition comprising a hyaluronidase and an effective amount of a second composition comprising a plasmin. In a further embodiment of this aspect of the invention, the first and second compositions may be administered to a subject at substantially the same time or at different times.

In a fifth aspect, the present invention provides a method of inducing posterior vitreous detachment comprising contacting the vitreous and/or aqueous humor with an effective amount of a composition comprising a hyaluronidase in combination with a plasmin.

In a sixth aspect, the present invention provides a method of inducing posterior vitreous detachment comprising contacting the vitreous and/or aqueous humor with an effective amount of a first composition comprising a hyaluronidase and an effective amount of a second composition comprising a plasmin. In a further embodiment of this aspect of the invention, the first and second compositions may be administered to a subject at substantially the same time or at different times.

In any of the first to sixth aspects of the invention described above, the step of contacting the vitreous and/or aqueous humor with a composition comprising a hyaluronidase can be performed as an adjunct to, or in the absence of vitrectomy.

In a seventh aspect, the present invention provides a method of performing a vitrectomy comprising the step of contacting the vitreous and/or aqueous humor with an effective amount of a composition comprising a hyaluronidase in combination with a plasmin. The contacting step can be performed at the same time as, or prior to vitrectomy.

In an eighth aspect, the present invention provides a method of performing a vitrectomy comprising the step of contacting the vitreous and/or aqueous humor with an effective amount of a first composition comprising a hyaluronidase and an effective amount of a second composition comprising a plasmin. In a further embodiment of this aspect of the invention, the first and second compositions may be administered to a subject at substantially the same time or at different times. The contacting step can be performed at the same time as, or prior to vitrectomy.

In a ninth aspect, the present invention provides a composition comprising at least one hyaluronidase in combination with at least one plasmin.

In a tenth aspect, the present invention provides a kit comprising a first composition comprising at least one hyaluronidase and a second composition comprising at least one plasmin.

In one embodiment of all aspects of the present invention, the methods of treatment or prevention of an eye disorder, or complications of an eye disorder, and methods of performing a vitrectomy result in the amelioration of an eye disorder by one or more of the following outcomes: reducing the viscosity of the vitreous, liquefying the vitreous, inducing posterior vitreous detachment, clearing or reducing hemorrhagic blood from the vitreous, vitreous cavity and/or aqueous humor, clearing or reducing intraocular foreign substances from the vitreous, vitreous cavity and/or aqueous humor, clearing or reducing materials toxic to the retina from the vitreous, vitreous cavity and/or aqueous humor, increasing the diffusion of an agent or a composition administered to the vitreous and/or aqueous humor, or reducing retinal neovascularization. In yet another embodiment of all aspects of the invention, the eye disorder or complication of an eye disorder sought to be treated or prevented is selected from the group consisting of retinal detachment, retinal tear, vitreous hemorrhage, diabetic vitreous hemorrhage, proliferative diabetic retinopathy, non-proliferative diabetic retinopathy, age-related macular degeneration, macular holes, vitreomacular traction, macular pucker, macular exudates, cystoid macular edema, fibrin deposition, retinal vein occlusion, retinal artery occlusion, subretinal hemorrhage, amblyopia, endophthalmitis, retinopathy of prematurity, glaucoma, retinitis pigmentosa, and any combination thereof.

Each of enzyme hyaluronidase and plasmin includes, but is not limited to, non-recombinant enzyme, recombinant enzyme, stabilized enzyme, and variants of enzyme, wherein the variants of enzyme include a catalytic domain of enzyme. Variants of enzyme include truncated forms of enzyme that can be produced by amino acid deletions from these proteins. All variants of enzyme are expected to have enzyme activity, even if they do not possess the same level of enzyme activity as enzyme. Variants of enzyme also include, but are not limited to, amino acid insertions and/or substitutions in these proteins. It is envisioned that amino acid substitutions made in enzyme are preferably conservative substitutions. Any variant of enzyme can be prepared by recombinant methods. Alternatively, variants of enzyme can be prepared by any other means well known in the art such as, but not limited to, digestion of non-recombinant enzyme. These variants of enzyme, or for that matter, the enzyme itself, can be assayed for enzyme activity. In addition, the variants of enzyme can be tested for their ability to induce PVD and/or effect vitreous liquefaction by injecting different doses of the variant in any balanced saline solution into porcine, feline, rabbit or post-mortem human eyes. If an enzyme combination can induce PVD and/or effect vitreous liquefaction in any of these eyes, that enzyme combination is considered to be useful for treating eye disorders of mammals. Preferably, the enzyme does not result in toxicity to the injected eye.

Enzyme can be prepared from non-recombinant enzyme. Alternatively, enzyme can be prepared by recombinant methods. Enzyme can be concentrated, stabilized, and/or lyophilized. Methods of concentrating proteins are well known to those of ordinary skill in the art. Stabilization is a method of protecting a protein from degradation and/or inactivation through the use of one or more stabilizing agents. Methods of lyophilizing proteins are well known to those of ordinary skill in the art. The lyophilized enzyme can be stored in vials (e.g., glass) in any amount, but preferably, in amounts that can be readily reconstituted for use.

Lyophilized enzyme can be reconstituted in an ophthalmologically acceptable carrier prior to being used for contacting the vitreous and/or aqueous humor. In one embodiment an ophthalmologically acceptable carrier is a sterile solvent having a pH and osmolarity that is compatible with the vitreous of the subject. Nonlimiting examples of ophthalmologically acceptable carriers are isotonic saline solution and balanced salt solution (BSS). A balanced salt solution typically contains: 0.64% sodium chloride, 0.075% potassium chloride, 0.048% calcium chloride dehydrate, 0.03% magnesium chloride hexahydrate, 0.39% sodium acetate trihydrate, 0.17% sodium citrate dehydrate, sodium hydride/hydrochloric acid to adjust the pH, and water.

The method of contacting the vitreous and/or aqueous humor using compositions comprising enzyme will depend upon the particular subject, the severity of the condition being treated and the dosage required for therapeutic efficacy, and can be determined by a physician on a patient-by-patient basis. Any method of contacting the vitreous and/or aqueous humor that provides an effective amount of enzyme to the vitreous and/or aqueous humor can be utilized. It should be understood that such contact with the vitreous and/or aqueous humor does not have to take place simultaneously with the administration of a composition comprising enzyme. The contact may be delayed or occur over an extended period of time from the time of administration. One method of contacting the vitreous and/or aqueous humor is by one or more intraocular injections directly into the vitreous and/or aqueous humor respectively. The vitreous and/or aqueous humor can also be contacted by sub-conjunctival, intramuscular or intravenous injections. Any of these injections can be provided using a liquid solution comprising enzyme according to procedures well known in the art. Alternatively, however, the vitreous and/or aqueous humor can be contacted with enzyme by any other suitable method, which results in sufficient distribution of enzyme to the vitreous and/or aqueous humor to treat or prevent the disorder, or a complication of a disorder, of the eye of a subject. A composition comprising enzyme can also be administered by placing an intra-vitreal implantable device. The present invention also envisions that the vitreous and/or aqueous humor can be contacted with enzyme using a depot, sustained release formulation, or any implantable device so that enzyme is supplied continuously.

Dosing regimens for enzyme can be readily determined by one of ordinary skill in the art and will vary depending on the patient and the effect sought. Enzyme can be used at any dose, which brings about desirable therapeutic effects, including but not limited to vitreous liquefaction, posterior vitreous detachment, and/or clearing of blood, toxic materials or foreign substances from the vitreous cavity, without causing significant toxicity to the eye (especially the retina) or associated anatomical structures. Additionally, enzyme may be administered as a single dose or in multiple doses.

The composition preferably contains nontoxic amounts of the active compounds in a pharmaceutically acceptable carrier. It is contemplated that plasmin in combination with hyaluronidase can be introduced into the vitreous at a dose such that substantially complete posterior vitreous detachment occurs without inflammation, retinal vascular constriction or hemorrhages, and without electroretinographic or histologic abnormalities. The dosages herein are expressed in terms of international units (IU). The hyaluronidase is dispersed in an ophthalmologically suitable carrier and is generally introduced into the vitreous of the eye at a dose of about 0.1 IU to about 1,000 IU, preferably, about 1 IU, 5 IU, 10 IU, 15 IU, 20 IU, 30 IU, 50 IU, 150 IU, 500 IU. The plasmin is dispersed in an ophthalmologically suitable carrier and is generally introduced into the vitreous of the eye at a dose of about 0.1 IU to about 50 IU, preferably, about 1 IU, 2 IU, 3 IU, 4 IU, 5 IU, 10 IU, 15 IU, 20, IU, 25 IU.

The amount of the active compounds introduced into the vitreous can vary depending on various factors. Low levels of the active compounds can be used to induce a slow rate of posterior vitreous detachment where the active compounds remain in the eye for long periods of time. Alternatively, high doses of the active compounds can be used to induce a rapid posterior vitreous detachment. For example, plasmin at doses of 4 IU and 6 IU can be introduced into the vitreous which induce posterior vitreous detachment in several hours. At higher doses of plasmin greater than about 20 IU, the plasmin can cause inflammation of the retina. Therefore, at higher dosages, it is generally preferred to introduce the hyaluronidase/plasmin composition for sufficient time to induce the desired extent of posterior vitreous detachment and then remove the vitreous and/or the composition from the eye to prevent or minimize inflammation of the retina or other abnormalities. Standard vitrectomy surgical techniques can be used to remove the vitreous from the eye and replace the vitreous with an ophthalmologically acceptable solution or composition to stabilize the ocular cavity. In other embodiments, the composition is left in the eye without performing the vitrectomy.

The process of the invention utilizes the combination of hyaluronidase and plasmin. The enzymes are found to have a synergistic effect when introduced into the eye in combination. Plasmin alone and hyaluronidase alone are not able to induce complete posterior vitreous detachment and cause inflammation of the retina at higher doses, while plasmin and hyaluronidase together are capable of inducing substantially complete posterior vitreous detachment at nontoxic doses.

A typical enzyme dosage is in the range of about 0.5 U to about 100 U per eye. If injected, enzyme can be provided in a delivery volume of about 0.05 ml to about 0.5 ml of a sterile solvent (e.g. sterile BSS) per eye. In those instances where a vitrectomy is to be performed, enzyme is left in the vitreous and/or aqueous humor for between about 15 and 120 minutes before removal of the vitreous. In one embodiment of the invention, a dose of 1 U of plasmin and 20 U of hyaluronidase is delivered in 0.1 ml of sterile BSS per eye. In another embodiment, a dose of 1 U of plasmin and 20 U of hyaluronidase is delivered in 0.1 ml of sterile BSS per eye for about 15-120 minutes prior to vitrectomy.

The present invention contemplates the use of a composition comprising a hyaluronidase in combination with a plasmin, or of a first composition comprising a hyaluronidase and a second composition comprising a plasmin. Accordingly, in one aspect of the invention, the vitreous and/or aqueous humor is contacted with a composition comprising enzyme. In one particular embodiment of this aspect of the invention, the vitreous and/or aqueous humor is contacted with a first composition comprising at least one hyaluronidase and with a second composition comprising at least one plasmin. The composition can be administered at substantially the same time or at different times. Furthermore, the vitreous and/or aqueous humor may be contacted with a composition comprising at least one additional agent. The additional agent is any protein (but not a hyaluronidase or plasmin), chemical or other substance that is useful in treating or preventing eye disorders, or complications of an eye disorder. Non-limiting examples of such additional agents usable with the present invention include glycosaminoglycanase enzymes such as chondroitinase ABC, chondroitinase AC, chondroitinase B, chondroitin 4-sulfatase, chondroitin 6-sulfatase and B-glucuronidase; collagenase enzymes; dispase; RGD containing peptides such as RGD, GRGDS, GRGDTP, Echistatin and Falvoridin; anti-integrin antibody; P2Y receptor antagonists; urea, hydroxyurea, thiourea and anti-angiogenic agents such as, but not limited to, vascular endothelial growth factor (VEGF) inhibitors (e.g., anti-VEGF antibodies, VEGF aptamers, soluble-VEGF receptors, etc.) and placental growth factor (P1GF) inhibitors (e.g., anti-P1GF antibodies, P1GF aptamers, soluble P1GF receptors, etc.). Most of these additional agents are themselves capable of promoting vitreous liquefaction and/or inducing posterior vitreous detachment. Anti-angiogenic additional agents could be useful in preventing neo-vascularization in the eye. Expression of VEGF and/or P1GF from a hypoxic retina is thought to result in the development of extraretinal neovascularization. Thus, inhibiting VEGF and/or P1GF would be an effective way to prevent neovascularization.

A composition comprising enzyme is useful to effect the liquefaction of the vitreous and/or the disinsertion or detachment of the vitreous from the retina and other tissues (e.g., epiretinal membranes, macula). As a result of this vitreous liquefaction and/or vitreous detachment, the tractional forces of the vitreous on the retina and other tissues are minimized and the rate of natural turnover of fluids within the vitreous is accelerated. Accordingly, compositions comprising enzyme are particularly suitable for the treatment or prevention of many disorders of the eye, which benefit from vitreous liquefaction, posterior vitreous detachment, decreasing extraretinal neovascularization and/or accelerated clearance of toxins or other deleterious substances (e.g., angiogenic factors, edema fluids, hemorrhagic blood etc.) from the posterior chamber of the eye and/or tissues adjacent to the posterior chamber, such as retina or macula). Examples of such eye disorders include, but are not limited to, retinal detachment, retinal tear, vitreous hemorrhage, diabetic vitreous hemorrhage, proliferative diabetic retinopathy, non-proliferative diabetic retinopathy, age-related macular degeneration, macular holes, vitreomacular traction, macular pucker, macular exudates, cystoid macular edema, fibrin deposition, retinal vein occlusion, retinal artery occlusion, subretinal hemorrhage, amblyopia, endophthalmitis, retinopathy of prematurity, glaucoma and retinitis pigmentosa, and others in which the clinical symptoms of these disorders respond to enzyme administration. The present invention contemplates the treatment of disorders of the eye comprising contacting the vitreous with a composition comprising enzyme. Such contact is expected to liquefy the vitreous and/or induce posterior vitreous detachment and/or clear the vitreous cavity of blood or other toxic substances and/or decrease extraretinal neovascularization, thereby treating or preventing the disorder.

The present invention is also directed to methods of preventing or inhibiting the onset of various disorders of the eye that are the result of, or exacerbated by, vitreous adhesion to the retina and vitreous contraction. In one embodiment, the methods of the present invention are able to prevent or inhibit the disorders, or complications resulting from a disorder in the eye of a subject without removing the vitreous from the eye. In particular, the invention is directed to a process of treating a patient with proliferative disorders or at risk of developing proliferative disorders, such as, but not limited to, a diabetic patient, by inducing posterior vitreous detachment as a prophylactic step in preventing or delaying the onset of disorders associated with vitreous contraction or neovascularization into the vitreous. In one embodiment of the invention, the composition is introduced into the eye of a diabetic patient to inhibit progression of diabetic retinopathy. Preferably, the composition is introduced into the eye before the proliferative disorders occur. In one embodiment, the composition is introduced into the vitreous of the eye before the onset of proliferative disorders and allowed to remain in the eye indefinitely without removing the vitreous from the eye. In further embodiments, the invention is directed to a process for inhibiting complications in central and branch retinal vein occlusion, such as retinal neovascularization and macular edema by inducing posterior vitreous detachment in a patient in need of such treatment. The present invention provides a process for treating impending or full-thickness macular hole (whether idiopathic or traumatic) by inducing posterior vitreous detachment. Preventing or reducing the incidence of retinal detachment, retinal tears and retinal hemorrhage caused by vitreous contraction can be achieved by inducing posterior vitreous detachment before such disorders occur and without removing the vitreous from the eye.

Many ophthalmic disorders have as a causative component, a destabilization of the blood-retina membrane. This destabilization permits various components (e.g., serum components, lipids, proteins) of the choriocapillaries to enter the vitreal chamber and damage the retinal surface. This destabilization is also a precursor to vascular infiltration of the vitreal chamber, known as neovascularization. Neovascularization of the vitreous is dependent on the matrix of the vitreous. Thus, liquefaction of the vitreous, which removes the matrix in the form of the polymerized vitreous, blocks neovascularization. In one embodiment, the invention provides a method of treating or preventing eye disorders by preventing or reducing the incidence of retinal neovascularization comprising contacting the vitreous with a composition comprising enzyme.

Several opthalmological disorders including diabetic retinopathy and trauma result in the rupture or leakage of retinal blood vessels with resultant bleeding into the vitreous (i.e., vitreous hemorrhage). Vitreous hemorrhage typically manifests as clouding or opacification of the vitreous and is sometimes, but not always, accompanied by tearing or detachment of the retina. In cases where the vitreous hemorrhage is accompanied by a retinal tear or detachment, it is important that such retinal tear or detachment be promptly diagnosed and surgically repaired. Failure to promptly diagnose and repair the retinal tear or detachment may allow photoreceptor cells of the retina, in the region of the tear or detachment, to become necrotic. Necrosis of the photoreceptor cells of the retina may result in loss of vision. Furthermore, allowing the retinal detachment to remain unrepaired for such extended period of time may result in further vitreous hemorrhage and/or the formation of fibrous tissue at the site of the hemorrhage. Fibrous tissue may result in the formation of an undesirable permanent fibrous attachment between the vitreous body and the retina. In the absence of any treatment, hemorrhagic clouding of the vitreous can take between 6-12 months or longer to clear sufficiently to allow trans-vitreal viewing of the retina. In such cases, where a physician would need to repair any part of the retinal surface, or where a physician would need to view the retinal surface of a patient that is prevented by an opaque or cloudy vitreous, a microsurgical procedure known as vitrectomy may need to be performed. This procedure involves removal of all or a portion of the vitreous with a microsurgical cutter and the replacement of the vitreous with a clear liquid or other substance that allows the ocular cavity to maintain its shape. Standard vitrectomy surgical procedures are well known to those of ordinary skill in the art. In one embodiment, the present invention contemplates contacting the vitreous with a composition comprising enzyme as an adjunct to vitrectomy. In other embodiments, the vitreous is contacted with the composition comprising enzyme in the absence of performing a vitrectomy.

The invention utilizes the combination of hyaluronidase and plasmin. The enzymes are found to have a synergistic effect. Plasmin alone and hyaluronidase alone are not able to achieve the medical effects, and can cause inflammation of the retina at higher doses.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

1. A method of treating or preventing a disorder, or a complication of a disorder, of the eye of a subject comprising contacting a vitreous and/or aqueous humor with an effective amount of a composition comprising a hyaluronidase in combination with a plasmin.

2. A method of treating or preventing a disorder, or a complication of a disorder, of the eye of a subject comprising contacting a vitreous and/or aqueous humor with an effective amount of a first composition comprising a hyaluronidase and an effective amount of a second composition comprising a plasmin.

3. A method of liquefying the vitreous body of a subject comprising contacting a vitreous and/or aqueous humor with an effective amount of a composition comprising a hyaluronidase in combination with a plasmin.

4. A method of liquefying the vitreous body of a subject comprising contacting a vitreous and/or aqueous humor with an effective amount of a first composition comprising a hyaluronidase and an effective amount of a second composition comprising a plasmin.

5. A method of inducing posterior vitreous detachment in an eye of a subject comprising contacting a vitreous and/or aqueous humor with an effective amount of a composition comprising a hyaluronidase in combination with a plasmin.

6. A method of inducing posterior vitreous detachment in an eye of a subject comprising contacting a vitreous and/or aqueous humor with an effective amount of a first composition comprising a hyaluronidase and an effective amount of a second composition comprising a plasmin.

7. A method of performing a vitrectomy in a subject comprising the step of contacting a vitreous and/or aqueous humor with an effective amount of a composition comprising a hyaluronidase in combination with a plasmin.

8. A method of performing a vitrectomy in a subject comprising the step of contacting a vitreous and/or aqueous humor with an effective amount of a first composition comprising a hyaluronidase and an effective amount of a second composition comprising a plasmin.

9. A composition comprising at least one hyaluronidase in combination with at least one plasmin.

10. A kit comprising a first composition comprising at least one hyaluronidase and a second composition comprising at least one plasmin.

11. The method of claim 1, wherein said hyaluronidase is non-recombinant hyaluronidase.

12. The method of claim 1, wherein said hyaluronidase is recombinant hyaluronidase.

13. The method of claim 1, wherein said plasmin is non-recombinant plasmin.

14. The method of claim 1, wherein said plasmin is recombinant plasmin.

15. The method of claim 1, wherein said hyaluronidase is a variant of hyaluronidase.

16. The method of claim 1, wherein said plasmin is a variant of plasmin.

17. The method of claim 1, wherein the disorder of the eye is selected from the group consisting of retinal detachment, retinal tear, vitreous hemorrhage, diabetic vitreous hemorrhage, proliferative diabetic retinopathy, non-proliferative diabetic retinopathy, age-related macular degeneration, macular holes, vitreomacular traction, macular pucker, macular exudates, cystoid macular edema, fibrin deposition, retinal vein occlusion, retinal artery occlusion, subretinal hemorrhage, amblyopia, endophthalmitis, retinopathy of prematurity, glaucoma, retinitis pigmentosa, and any combination thereof.

18. The method of claim 1 to effect an outcome, wherein the outcome is selected from the group consisting of reducing the viscosity of the vitreous, liquefying the vitreous, inducing posterior vitreous detachment, clearing or reducing hemorrhagic blood from the vitreous and/or aqueous humor, clearing or reducing intraocular foreign substances from the vitreous and/or aqueous humor, clearing or reducing materials toxic to the retina from the vitreous and/or aqueous humor, increasing the diffusion of an agent or a composition administered to the vitreous and/or aqueous humor, reducing retinal neovascularization, and any combination thereof.

19. The method of claim 1, further comprising contacting said vitreous and/or aqueous humor with an effective amount of a composition comprising chondroitinase, collagenase, dispase, RGD containing peptides, anti-integrin antibody, P2Y receptor antagonists, urea, hydroxyurea, thiourea, angiogenic inhibitors, VEGF inhibitors, P1GF inhibitors, and any combination thereof.

20. The method of claim 1, wherein the composition is a liquid solution, and wherein the step of contacting the vitreous and/or aqueous humor with the composition comprises injecting the solution into the vitreous and/or aqueous humor.
